(19) United States
(12) Patent Application Publication
(10) Pub. No.: US 2016/0003842 A1
(43) Pub. Date: Jan. 7, 2016

(54) GLYCOPEPTIDE IDENTIFICATION

(71) Applicant: CHILDREN'S MEDICAL CENTER CORPORATION, Boston, MA (US)

(72) Inventors: Richard S. Lee, Needham, MA (US); John Froehlich, Jamaica Plain, MA (US)

(73) Assignee: Children's Medical Center Corporation, Boston, MA (US)

(21) Appl. No.: 14/768,970

(22) PCT Filed: Feb. 20, 2014
(86) PCT No.: PCT/US2014/017311

§ 371 Date: Aug. 19, 2015

Related U.S. Application Data
(60) Provisional application No. 61/767,735, filed on Feb. 21, 2013.

(51) Int. Cl. G01N 33/68; H01J 49/00

(52) U.S. Cl. CPC G01N 33/6848; H01J 49/0045; H01J 49/0036; G01N 2400/00

(57) ABSTRACT

A system including a device with at least one processor and memory storing computer-executable instructions that, when executed by the at least one processor, perform a method of identifying glycopeptides in a sample, the method including, analyzing a mass spectrum of the sample to identify at least one portion of the mass spectrum having at least one characteristic of mass spectra indicative of presence of glycopeptides, identifying the glycopeptides in the sample based on the at least one identified portion; and analyzing at least one glycopeptide of the identified glycopeptides.

### BACKGROUND

As mass spectrometric (MS) techniques become increasingly available and accessible, large variety of molecules can be analyzed using this approach. MS techniques generate data about masses of molecules and their intensities for a particular scan. A mass spectrometer is a device that separates and quantifies ions based on their mass to charge (m/z) ratios. In the tandem MS, also referred to as MS/MS, a particular ion is fragmented and a mass spectrum of the fragments is generated. The ion that is fragmented may be referred to as the “precursor” and the ions in the tandem-MS spectrum may be called “products.” MS, liquid chromatography MS (LC-MS), LC-MS/MS and other variations of mass spectrometry techniques have been used in proteomics, particularly, in the analysis of glycoproteins and glycopeptides. Glycopeptides are peptides that include carbohydrate moieties (glycans) covalently attached to the side chains of the amino acid residues that constitute the peptide. Glycoproteins play important roles in fertilization, the immune system, brain development, the endocrine system and inflammation. Moreover, glycopeptides have been utilized in therapeutic applications. Cell surface proteins of human cells can be markers of disease. N-glycosylation is a post-translational modification which affects cell-cell signaling, protein stability, and has been implicated in various pathologies. (Varki, 1993).

Accordingly, determining which glycan moieties occupy specific glycosylation sites and characterizing glycan heterogeneity is required for understanding of the biological roles of glycoproteins, as well as for assuring correct glycosylation on glycoprotein therapeutics. (Kolarich et al., 2012). However, accurate analysis of glycoprotein site occupancy and glycan heterogeneity may be a challenging task.

### SUMMARY

Techniques are provided that allow generating glycopeptide spectral data which may be analyzed for the presence of glycopeptides. A tool is implemented that may provide a glycopeptide spectral profile for a biological sample. The tool may allow discriminating between peptides and glycopeptides in complex mixtures of biological origin based on accurate mass measurements of precursor peaks. With the growing availability of mass analyzers, such as, for example, high mass accuracy mass analyzers, the described approach represents a simple and broadly applicable way of increasing accuracy and sensitivity of MS/MS-based glycoproteomic analyses.

The tool may discriminate between peptides and glycopeptides based on fractional mass values (mass defects) of the elements in a sample and may thus be used in diverse glycoproteomic applications, without the need for prior knowledge regarding the analyzed proteome or glycome. The tool may be based on identification of glycopeptide-rich acquisition enhancement zones (GRAEZs) and may be referred to as GRAEZ classifier. The GRAEZ classifier may be used, for example, to compare the effectiveness of different glycopeptide sample preparations. Further, GRAEZ classification of existing proteomic data sets may be used to evaluate the prevalence of glycosylated peptides in existing data. This may improve accuracy and sensitivity of analysis of glycoproteome in biological samples.

In some embodiments, the tool may operate in association with any suitable glycopeptides identification software and may increase accuracy, sensitivity and specificity of such software. Furthermore, the tool may be incorporated into any MS analyzer to make it possible for the analyzer to accurately identify glycopeptides, which may be performed in real time.

According to an embodiment, there is provided at least one computer-readable storage medium storing computer-executable instructions that, when executed by at least one processor, perform a method of identifying glycopeptides in a sample, the method comprising analyzing a mass spectrum of the sample to identify at least one portion of the mass spectrum having at least one characteristic of mass spectra indicative of presence of glycopeptides; and identifying the glycopeptides in the sample based on the at least one identified portion.

According to an embodiment, the method further comprises determining the at least one characteristic of mass spectra indicative of presence of glycopeptides.

According to an embodiment, determining the at least one characteristic comprises determining at least one glycopeptide-rich acquisition enhancement zone.

According to an embodiment, determining the at least one characteristic comprises analyzing a training data set comprising a plurality of mass spectra of peptides.

According to an embodiment, the at least one characteristic comprises at least one first range of a nominal mass and at least one second range of mass defect.

According to an embodiment, the method further comprises displaying on a user interface results of the identification of the glycopeptides in the sample.

According to an embodiment, displaying the results of the identification of the glycopeptides comprises displaying the results so that the glycopeptides in the sample are differentiated from peptides in the sample.

According to an embodiment, the method further comprises providing a representation of the results of the identification of the glycopeptides so that the representation is enabled to receive input indicating selection of at least one glycopeptide of the identified glycopeptides for further analysis

According to an embodiment, the method further comprises further analyzing the at least one glycopeptide selected for the further analysis.

According to an embodiment, identifying the glycopeptides in the sample comprises identifying N-glycosylated glycopeptides.

According to an embodiment, the method further comprises providing results of the identification of the glycopeptides in the sample to a system configured to further analyze the identified glycopeptides.

According to an embodiment, the method further comprises further analyzing at least one of the identified glycopeptides.

According to an embodiment, the sample comprises a biological sample.

According to an embodiment, the biological sample is obtained from tissue, urine, blood, plasma, serum or saliva.

According to an embodiment, the at least one characteristic is determined for a protease used to generate a mixture of peptides and glycopeptides from the sample.

According to an embodiment, analyzing the mass spectrum comprises analyzing precursor ion data.

According to an embodiment, at least one computer-readable storage medium storing computer-executable instructions that, when executed by at least one processor, perform a method of identifying glycopeptides in a sample, the method comprising determining at least one characteristic of mass spectra indicative of presence of glycopeptides; analyzing a mass spectrum of the sample to identify at least one portion of the mass spectrum having the at least one characteristic; and identifying the glycopeptides in the sample based on the at least one identified portion.

According to an embodiment, there is provided a computer-implemented method of identifying glycopeptides in a sample, the method comprising at least one processor, analyzing a mass spectrum of the sample to identify at least one portion of the mass spectrum having at least one characteristic of mass spectra indicative of presence of glycopeptides; and identifying the glycopeptides in the sample based on the at least one identified portion.

According to an embodiment, the method further comprises determining the at least one characteristic of mass spectra indicative of presence of glycopeptides.

According to an embodiment, determining the at least one characteristic comprises determining at least one glycopeptide-rich acquisition enhancement zone.

According to an embodiment, determining the at least one characteristic comprises analyzing a data set comprising a plurality of mass spectra of peptides to determine at least one first range of a nominal mass and at least one second range of mass defect indicative of presence of glycopeptides

According to an embodiment, determining the at least one characteristic comprises analyzing a training data set comprising a plurality of mass spectra of peptides.

According to an embodiment, the at least one characteristic comprises at least one first range of a nominal mass and at least one second range of mass defect.

According to an embodiment, the method further comprises displaying on a user interface results of the identification of the glycopeptides in the sample.

According to an embodiment, displaying the results of the identification of the glycopeptides comprises displaying the results so that the glycopeptides in the sample are differentiated from peptides in the sample.

According to an embodiment, the method further comprises providing a representation of the results of the identification of the glycopeptides so that the representation is enabled to receive input indicating selection of at least one glycopeptide of the identified glycopeptides for further analysis

According to an embodiment, the method further comprises further analyzing the at least one glycopeptide selected for the further analysis.

According to an embodiment, the method further comprises further analyzing the at least one glycopeptide selected for the further analysis comprises determining a site of glycosylation on the at least one glycopeptide.

According to an embodiment, determining the site of glycosylation comprises determining a site of N-glycosylation on the at least one glycopeptide.

According to an embodiment, the method further comprises analyzing the at least one glycopeptide using tandem mass-spectrometry.

According to an embodiment, identifying the glycopeptides in the sample comprises identifying N-glycosylated glycopeptides.

According to an embodiment, the method further comprises providing results of the identification of the glycopeptides in the sample to a system configured to further analyze the identified glycopeptides.

According to an embodiment, the method further comprises further analyzing at least one of the identified glycopeptides.

According to an embodiment, the sample comprises a biological sample.

According to an embodiment, the biological sample is obtained from tissue, urine, blood, plasma, serum or saliva.

According to an embodiment, analyzing the mass spectrum comprises analyzing precursor ion data.

According to an embodiment, there is provided a device comprising at least one processor and memory storing computer-executable instructions that, when executed by the at least one processor, perform a method of identifying glycopeptides in a sample, the method comprising analyzing a mass spectrum of the sample to identify at least one portion of the mass spectrum having at least one characteristic of mass spectra indicative of presence of glycopeptides; and identifying the glycopeptides in the sample based on the at least one identified portion.

According to an embodiment, the method further comprises determining the at least one characteristic of mass spectra indicative of presence of glycopeptides.

According to an embodiment, determining the at least one characteristic comprises determining at least one glycopeptide-rich acquisition enhancement zone.

According to an embodiment, determining the at least one characteristic comprises analyzing a data set comprising a plurality of mass spectra of peptides to determine at least one first range of a nominal mass and at least one second range of mass defect indicative of presence of glycopeptides.

According to an embodiment, determining the at least one characteristic comprises analyzing a training data set comprising a plurality of mass spectra of peptides.

According to an embodiment, there is provided a device comprising at least one processor and memory storing computer-executable instructions that, when executed by the at least one processor, perform a method of identifying glycopeptides in a sample, the method comprising analyzing a mass spectrum of the sample to identify at least one portion of the mass spectrum having at least one characteristic of mass spectra indicative of presence of glycopeptides; identifying the glycopeptides in the sample based on the at least one identified portion; and analyzing at least one glycopeptide of the identified glycopeptides.

According to an embodiment, the method further comprises determining the at least one characteristic of mass spectra indicative of presence of glycopeptides.

According to an embodiment, determining the at least one characteristic comprises determining at least one glycopeptide-rich acquisition enhancement zone.

According to an embodiment, determining the at least one characteristic comprises analyzing a data set comprising a plurality of mass spectra of peptides to determine at least one first range of a nominal mass and at least one second range of mass defect indicative of presence of glycopeptides.

According to an embodiment, determining the at least one characteristic comprises analyzing a training data set comprising a plurality of mass spectra of peptides.

According to an embodiment, analyzing the at least one glycopeptide comprises determining a site of glycosylation on the at least one glycopeptide.

According to an embodiment, determining the site of glycosylation comprises determining a site of N-glycosylation on the at least one glycopeptide.

### BRIEF DESCRIPTION OF THE DRAWINGS

FIG. 1 is a conceptual overview of mass defect classification of glycopeptides. Initial glycopeptide enrichment is followed by a LC-MS or LC-MS/MS analysis. After peak picking and deconvolution, a list of monoisotopic m/z values and retention times is generated. This list is then sorted into likely glycopeptide and likely peptide precursors on the basis of accurate mass. Targeted LC-MS/MS analysis is then possible without prior proteomic or glycomic characterization.

FIGS. 2A and 2B illustrate a mass defect plot of the Tryptic (A) and chymotryptic (B) in silico digests. Peptides are plotted in dark grey (blue) and labeled with a numerical reference 202; glycopeptides are plotted in light grey (green), the GRAEZ boundaries are delineated by black lines and the GRAEZ regions are labeled with a numerical reference 200. FIG. 2A shows tryptic digests. FIG. 2B shows chymotryptic digests. There is a shift in mass defect (y-axis) between peptides and glycopeptides of a given nominal mass (x-axis). This shift may be observed for each protease treatment; the optimal GRAEZ settings may be distinct for each protease treatment.

FIGS. 3A and 3B illustrate examples of two glycopeptide MS/MS spectra. FIG. 3A shows a complex, monosialylated, difucosylated N-glycan observed. FIG. 3B shows a complex monosialylated N-glycan observed. Fragment ions are observed as a series of Y-type ions from the intact N-glycopeptide precursor and a clear sequential loss of the N-linked core mannoses and N-acetylglucosamine. In each case, a ^{0.2}X₀ type cleavage is observed for the reducing end N-acetylglucosamine. Remaining glycan compositions are assigned by accurate mass losses from the precursor ion, and a minimum of four Y- or X-type ions were required for each assignment. A predicted glycan is shown for each spectrum which reflects the composition determined.

FIGS. 4A and 4B illustrate plots of size distributions for tryptic and chymotryptic peptides.

FIG. 5 illustrates an exemplary computing environment in which some embodiments may be implemented.

### DETAILED DESCRIPTION

The inventors have appreciated that existing approaches to identification of glycopeptides in biological samples may lack adequate accuracy and sensitivity that would make the approaches useful for practical applications of proteomics. For example, analysis of site-specific N-glycosylation may be complicated. Such analysis may not be easily accomplished because of heterogeneity at the levels of glycosylation site occupancy, glycan composition, and glycan structure. A comprehensive analysis of protein glycosylation identifies glycans, maps occupied sites, and matches the glycans to specific sites on glycoproteins. (An et al., 2009). This site-specific analysis may be performed via analysis of intact glycopeptides using mass spectrometry (MS). However, this technique may be complicated by sensitivity, sample preparation, and fragmentation challenges (Dodds, 2012), which may limit the throughput and sensitivity of the results.

The site-specific glycosylation analysis may be complicated by the presence of nonglycosylated peptides in a mixture, as they may be preferentially selected for data-dependent MS/MS due to higher ionization efficiencies and higher stoichiometric levels in samples.

Some of the approaches to determining which glycan moieties occupy specific N-glycosylation sites include liquid chromatography MS (LC-MS) and LC-MS/MS analysis of glycopeptides generated by proteases with high cleavage site specificity; however, a sensitivity achieved by such approach may be limited.

Furthermore, the analysis of site-specific glycosylation may be complicated because the ionization of glycopeptides is suppressed by any nonglycosylated peptides which are coproduced during protease digestion with specific proteases. As an alternative approach, digestion using nonspecific proteases has been implemented to eliminate competing peptide species. (Dalpathado et al., 2006; Clowers et al., 2007). Specific proteases may yield predictable peptide footprints, and have been utilized for analysis of complex mixtures. However, glycopeptides are often not selected for fragmentation in data-dependent analysis (DDA) (Kolarich et al., 2012), making glycopeptide identification unfeasible, as fragmentation is required for glycopeptide identification in samples. (Desaire and Hua, 2009). To circumvent this shortcoming, glycopeptide enrichment protocols using normal-phase, HILIC, or lectin enrichment techniques have been established to enrich for glycopeptides. (Ito et al., 2009). However, these purification approaches have varying specificities for glycopeptides, may preferentially isolate glycopeptides with certain types of glycans attached, and add additional sample handling steps.

The inventors have recognized and appreciated that a classifier capable of discriminating between peptide and glycopeptide signals in mass spectrometry may improve accuracy and sensitivity of glycoproteomics analysis and may facilitate various purification techniques now known and developed in the future. In MS/MS, a precursor ion dissociates to a smaller fragment ion as a result of collision-induced dissociation.

Accordingly, in some embodiments, a tool is provided that may facilitate discrimination between peptides and glycopeptides in a sample based on accurate mass measurements of precursor ion peaks. The mass measurements may be performed using any suitable mass analyzer—for example, a high mass accuracy mass analyzer may be utilized. Any suitable sample comprising a complex mixture of biological origin may be analyzed. For example, the sample may be a biological sample obtained, for example, from tissue, blood, urine, plasma, serum, or any other biological sample.

The described techniques may be implemented as a tool that may be used to analyze proteomic data to discriminate between glycopeptides (e.g., N-glycopeptides) and nonglycosylated peptides based on accurate mass measurements. The tool is based on determining glycopeptide-rich acquisition enhancement zones (GRAEZs) and may be referred to by way of example as GRAEZ classification or a GRAEZ classifier. It should be appreciated that embodiments of the disclosed technology are not limited to a particular way of referring to the tool.

The described techniques may be implemented as software, hardware, firmware, circuitry, or a combination thereof. In some embodiments, the tool may be implemented as computer-executable instructions stored on one or more computer-readable storage media. The computer-executable instructions, when executed by at least one processor, may perform the method of analyzing a sample to discriminate between glycopeptides and peptides. The computer-executable instructions may be executed on any suitable computing device, as embodiments of the disclosed technology are not limited in this respect. Furthermore, the tool may be implemented in hardware, or any suitable combination of software and hardware, and embodiments of the disclosed technology are not limited to a particular way of implementing the tool.

Furthermore, the described techniques may be incorporated into any suitable system or device. For example, the tool may be incorporated into a system or device performing data-dependent acquisition (DDA), which may be defined as a mode of data collection in tandem mass spectrometry in which a number of peaks selected from an initial (or survey) scan using predetermined rules are selected and the corresponding ions are subjected to MS/MS analysis. Performance of such DDA systems, which may be referred to by way of example as DDA engines, may be improved by using the tool, since more accurate identification of glycopeptides in biological samples may be achieved.

By using the tool, in some embodiments, glycopeptides which were not fragmented in an initial data-dependent acquisition analysis of a sample run may be targeted in a subsequent analysis without any prior knowledge of glycans or proteins present in the sample. Furthermore, molecular species identified to likely be glycopeptides and which were not sufficiently fragmented in an initial analysis may be reacquired using glycopeptide settings of the tool.

Fragment ions have been found which are specific to glycopeptides. (Huddleston et al., 1993; Jebanathirajah et al., 2003). However, these may not be useful if the glycopeptides were not selected for fragmentation, or if they yield low quality MS/MS spectra. As mass defect (MD) classifications have been applied to similar challenges in proteomics (Bruce et al., 2006; Dodds et al., 2006; Kirchner et al., 2010;), the inventors determined whether a MD classification may be useful for discriminating between peptides and glycopeptides.

A lower MD has been observed for glycopeptides, due to a relative increase of oxygen (and its negative MD value) in glycopeptides. (Lehmann et al., 2000). However, this observation was made through comparison of tryptic peptides and small glycopeptides generated by nonspecific proteolysis.

Accordingly, the inventors have recognized and appreciated that it may be useful to utilize the MD shift associated with the relative increase of oxygen in glycopeptides to develop a classification approach implemented by the tool. The inventors determined true positive rates (TPR) and false positive rates (FPR) of the GRAEZ classifier based on accurate mass measurements. Furthermore, it was evaluated whether the MD shift may be observed for peptides and glycopeptides generated by the same protease (e.g., when conventional sample preparation protocols are utilized).

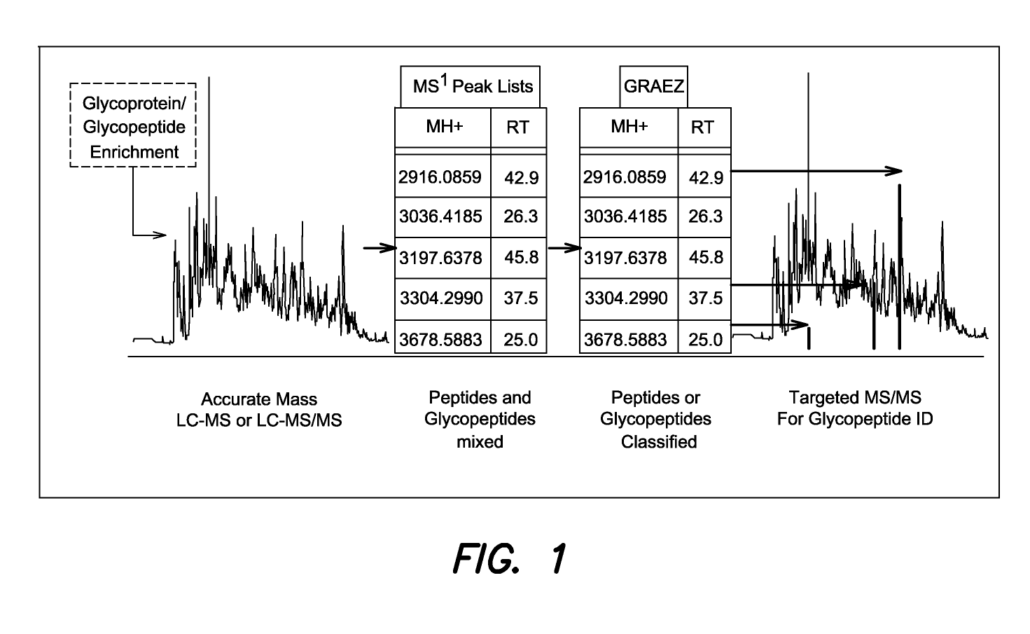

Accordingly, the glycopeptide-rich acquisition enhancement zones were determined and their utility in identifying precursor m/z values useful for large-scale glycopeptide assignment by tandem MS was evaluated. This classification may be applied to identify likely glycopeptides (e.g., N-glycopeptides) without parallel proteomic or glycomic experiments and without any prior knowledge of the proteome or glycome present in an analyzed sample. Targeted MS studies of molecular species using the tool described herein may increase selection of glycopeptides for fragmentation and thus improve efficiency and accuracy of glycopeptide identification and characterization. This concept is shown schematically in FIG. 1. Also, the efficacy of GRAEZ classification performed using the tool was demonstrated by validating the classifier on an LC-MS/MS data from urinary proteomics analysis.

The tool described herein may be useful in a wide range of applications. For example, manufacturers of therapeutic glycoproteins may use the tool to determine the microheterogeneity of glycosylation on a therapeutic with improved accuracy. This may be particularly useful for therapeutics with more than one site of glycosylation.

Further, the tool may be used to evaluate efficacy and stability of different glycoforms of therapeutic glycoproteins, evaluate changes in binding affinity of a therapeutic for an individual patient, based on the glycosylation of native receptors of interest. The tool may also be applicable in personalized medicine approaches where drug efficacy or treatment decisions may be made based on the glycan microheterogeneity of specific glycoproteins of interest. Glycoprotein microheterogeneity or changes in glycoprotein microheterogeneity may be analyzed using the tool in applications related to specific drug treatments, infection, disease/biomarker discovery, development, signaling, immunological disorders, immunoreactivity, ageing and any other applications.

Methods

The GRAEZ MD settings were determined using an in silico training data set and evaluated using an in silico test data set of peptides and glycopeptides. Training and test sets were generated from the HUPO plasma proteome database, which may be accessed at http://www.peptideatlas.org/hupo/hppp/. Entries were re-mapped to SwissProt Identifiers using an online tool (www.uniprot.org). A total of 1797 unique entries were generated. Six hundred random protein entries were selected and digested in silico with either trypsin or chymotrypsin using MS-Digest (www.prospector.ucsf.edu) to form the training sets. The remaining 1197 proteins were used to form the test set. By way of example, one missed cleavage was permitted, cysteine residues were considered as their carbamidomethyl derivatives, and peptide output was selected to be more than three amino acids and 400-5000 Daltons. This range was chosen by way of example as comprising peptide sizes that may be analyzed using conventional MS analyzers. Though, it should be appreciated that embodiments of the disclosed technology are not limited to a particular range for peptides, and other ranges may be substituted. MS-Digest reported singly protonated m/z values for all peptides.

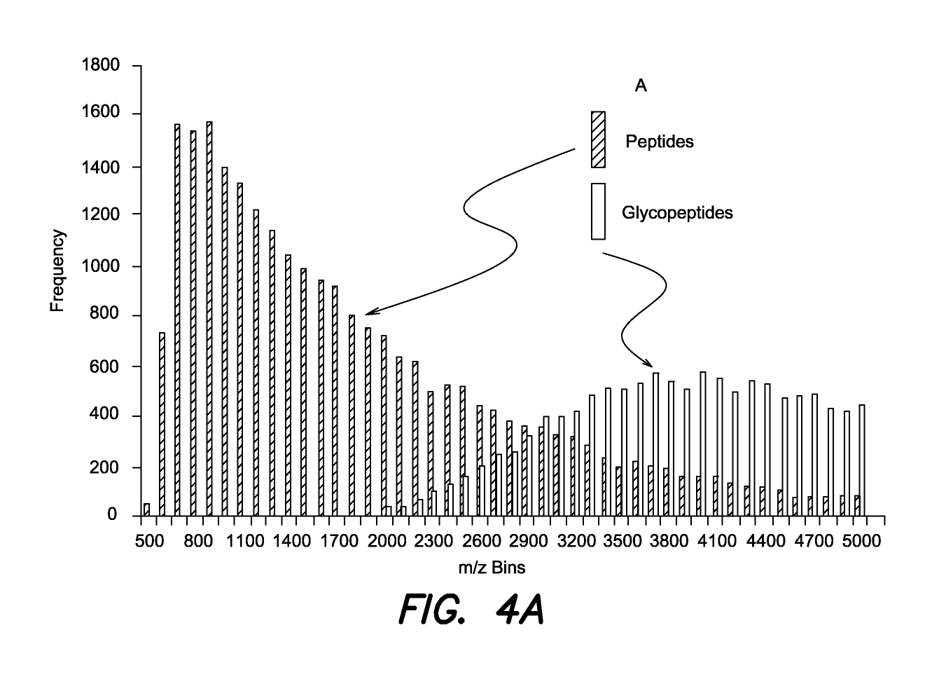

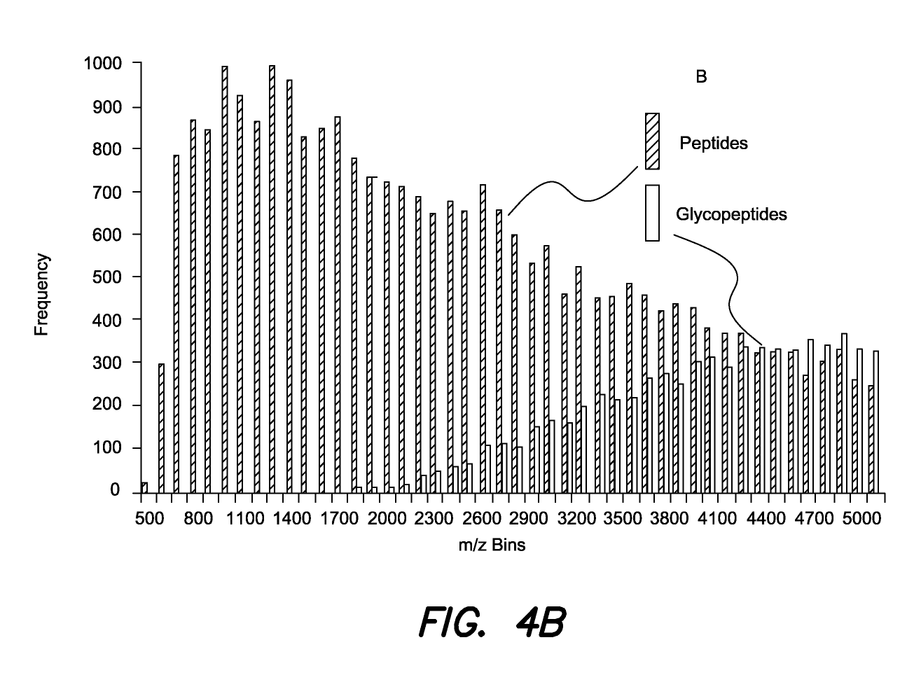

In some embodiments, redundant peptide sequences were removed. Peptides containing potential N-glycosylation consensus sites (CS pepti disclosed technology des) were identified by the presence of NXS or NXT sequences, where X is any amino acid except proline. Glycopeptides were then generated in silico by adding the monosaccharide masses of eight distinct N-glycan compositions to each CS peptide. The glycans utilized are shown in Table 1 and were chosen to represent common Homo sapiens N-glycans, without biasing the classifier for large N-linked glycans excessively. Since the MD shift is proportionally less for smaller N-glycans, a range of N-glycan masses was tested to challenge the classifier. Size distributions for tryptic and chymotryptic peptides are shown in FIGS. 4A and 4B, respectively.

TABLE 1
Eight relevant N-glycans utilized to generate glycopeptides in silico.

| N-Glycan ID | Hex | HexNAc | Fuc | SA | Mass Added |
|---|---|---|---|---|---|
| Glycan 1 | 5 | 2 | | | 1216.4228 |
| Glycan 2 | 7 | 2 | | | 1540.5284 |
| Glycan 3 | 9 | 2 | | | 1864.634 |
| Glycan 4 | 5 | 4 | | | 1622.5816 |
| Glycan 5 | 5 | 4 | 2 | | 1914.6974 |
| Glycan 6 | 5 | 4 | | 2 | 2204.7724 |
| Glycan 7 | 6 | 5 | 2 | | 2279.8296 |
| Glycan 8 | 6 | 5 | | 2 | 2569.9046 |

Abbreviations used:
Hex (hexose),
HexNAc (N-acetyl hexosamine),
Fuc (deoxyhexose),
SA (N-acetylneuraminic acid).
Mass added is equal to the increase in the monoisotopic mass of peptides when the N-glycan is added.

Peptides and glycopeptides were plotted on a mass defect (MD) map to identify initial trends in integer and defect mass for each species, and best-fit lines were generated for each class. Initial GRAEZ settings were set between the best-fit lines for each class, and the accuracy (or % of correct assignments) of the classifier was evaluated. The initial slope and intercept values were then optimized using an automated iterative process to maximize accuracy.

Results

The conducted experiments demonstrated high sensitivity (0.892) and specificity (0.947) based on an in silico dataset comprising over 100,000 tryptic species. Comparable results were obtained using chymotryptic species. Further validation using existing data and a fractionated tryptic digest of human urinary proteins was performed, yielding a sensitivity of 0.90 and a specificity of 0.93.

Precursors within the GRAEZ may be enriched in glycopeptides—e.g., by an order of magnitude. The tool allows identifying an N-glycopeptide-enriched targeted list from an initial data-dependent analysis to thus efficiently target glycopeptides in a subsequent analysis. The tool, which may be implemented in software executed on a computing device, may be “trained” to select likely glycopeptide masses for MS/MS.

For analysis using the tool, no prior information about an analyzed sample may be required. Thus, no glycomic or proteomic experiments may need to be performed. The analysis using the tool may be performed after glycopeptide enrichment, thus decreasing peptide contamination and improving the outcome of glycopeptide enrichment approaches by increasing glycopeptide sampling in MS/MS analysis. Moreover, the analysis may be performed after an initial proteomics DDA analysis, resulting in extensive coverage of glycopeptide targets.

### EXAMPLES

To retrospectively verify the in silico findings, a catheterized urine sample from a healthy male infant was obtained with an IRB-approved protocol and processed using a previously published sample preparation method for urinary proteomics. (Vaezzadeh et al., 2010). Urine was concentrated and desalted on 5K MWCO spin filters (Sartorius). Proteins were reduced and alkylated in the spin filter, washed extensively with TEAB, and removed from the upper chamber before digestion with trypsin at a (w/w) ratio of 50:1 sample:enzyme overnight at 37° C. Peptides were labeled with TMT⁶-126 (Thermo) according to manufacturer's instructions, and purified with HLB cartridges (Oasis). Peptides were separated into 24 fractions using an Agilent OFFGEL isoelectric point fractionator for 50 kVh, extracted, and dried.

Individual fractions were reconstituted in loading buffer and analyzed by LC-MS/MS using a Thermo QExactive MS system equipped with an eksigent 2D nano LC system, autosampler, and C₁₈ column (15 cm length by 17 micron diameter). A “top 10” data dependent LC-MS/MS method was utilized, resolution was set to 70K for MS¹ and 17.5 K for MS² scans. A 60 minute linear gradient from 5%-35% ACN was used. Normalized collision energy was 30 and the AGC was set for 1e⁶ for MS¹ and 5e⁴ for MS² scans.

In addition to the retrospective GRAEZ evaluation, prospective GRAEZ testing was also performed. Tryptic peptides were generated as above using a urine sample donated by a healthy male adult. An initial DDA run was performed on the non-fractionated sample after cleanup. After acquisition, all MS¹ features were extracted using Maxquant (Cox et al., 2008) and evaluated for GRAEZ status. A list of 2,325 unique precursors was generated which were classified as glycopeptides by GRAEZ, and targeted in two subsequent LC-MS runs. Data were acquired with similar instrumental parameters, except the normalized collision energy was 29 and the AGC was set for 3e⁶ for MS¹ and 1e⁵ for MS² scans.

All MS² spectra from the retrospective experiment were searched for the presence of two marker ions, the TMT reporter ion at 126.1277 Daltons, or the diagnostic Hex₁HexNac₁ oxonium ion at 366.1395. Prospective data were evaluated for the 366.1395 and 204.0867 ions. Rapid identification of the relevant precursor m/z and z values was achieved by the use of an in-house script which functions as an add-in for the msconvert tool. The tool, mzpresent, filters all MS² spectra for user-defined fragment ions and creates an mgf file and a comma separated value file as output which contains scan number, retention time, m/z selected for fragmentation, charge state of the precursor, and the intensity of the fragment ion. mzPresent is available for download at http://softwaresteenlab.org/, and may use any arbitrary m/z value.

By way of example, 10 (parts-per-million) ppm mass error was allowed and a minimum of 25% relative intensity was required for the fragment ions. The precursor m/z and z values were used to calculate (M+H)+ values for GRAEZ classification, and these GRAEZ classifications were cross-referenced against the presence of the glycopeptide-specific ions tandem (MS²) spectra to estimate the TPR/FPR ability of GRAEZ.

### Creating GRAEZ Settings and in Silico Evaluation

Due to the contribution of N-linked glycans, N-glycopeptides are typically larger in size than peptides. Based on the in silico data, all species below 1500 Daltons were thus excluded from targeted N-glycopeptide analysis with negligible loss in sensitivity. Approximately 49% of tryptic peptides and 43% of chymotryptic peptides were smaller than 1500 Daltons. However, the in silico specificity measures listed below do not consider the elimination of these low-mass species and therefore are quite conservative with regard to overall glycopeptide specificity.

An example of GRAEZ settings are shown below, where NM is the nominal mass (i.e., integer portion of the mass) of the singly protonated (or multiply protonated and deconvoluted) species being tested and MD is the defect mass (i.e., decimal portion of the mass). Species within the GRAEZ regions, or boundaries, are more likely to be glycosylated peptides, as discussed in more detail below.

0.000527(NM)−0.2204>MD>0.0003408(NM)+0.0219{NM<2316; 2870<NM<4214}

0.000527(NM)−0.2204>MD or

>0.0003408(NM)+0.219{2315<NM<2871; 4213<NM<5001} Trypsin GRAEZ

0.0005427(NM)−0.2641>MD>0.0003816(NM)−0.1031{NM<2350; 2890<NM<4172}

0.0005427(NM)−0.2641>MD or

>0.0003816(NM)−0.1031{2349<NM<2891; 4171<5001} Chymotrypsin GRAEZ

Results of the analysis of a sample using the tool may be represented on a user interface in any suitable manner. Accordingly, user experience may be improved when the results are visualized. The user interface may be presented on any suitable display. Though, it should be appreciated that embodiments of the disclosed technology are not limited to any particular way of reporting results of the analysis performed using the tool.

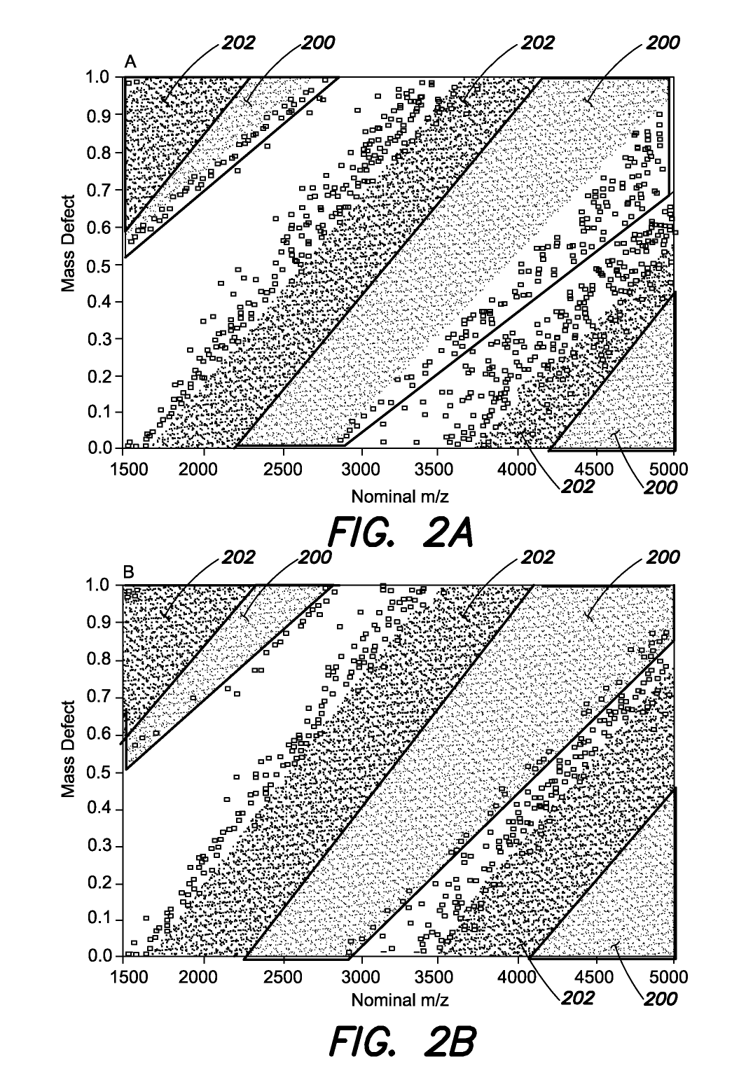

The GRAEZ regions determined by the above equations are shown in FIGS. 2A and 2B with the numerical reference 200 and the boundaries of the GRAEZ regions are delineated by black lines. In FIGS. 2A and 2B, peptides are plotted in dark grey (blue) and labeled with the numerical reference 202, and glycopeptides are plotted in light grey (green).

The “or” conditions shown above may be used when the calculated values for the “high” end of the GRAEZ becomes greater than 1 or greater than 2. Any calculated GRAEZ values which were larger than 1 had their integer value subtracted, as MD by definition is between the values of 0 and 1. A species which satisfies the condition may be classified as a glycopeptide by GRAEZ. For example, a tryptic species with a deconvoluted (M+H)⁺ value of 3449.4392 Daltons would fall between NM 2870 and 4214, and be evaluated as:

Decimal Value((0.0005247)×(3449)−(0.2204))=0.5892>0.4392>Decimal

Value((0.0003408)×(3449)+(0.0219))=0.197; GRAEZ glycopeptides.

For a large-scale analysis, GRAEZ testing may be performed on a suitable platform after deconvolution of LC-MS data.

The tryptic training set had a sensitivity of 0.952 and a specificity of 0.900 within the mass range of 1500 to 5000 Daltons. After eliminating m/z values outside the GRAEZ (or GRAEZing for glycopeptides), the glycopeptide: peptide ratio increased 9.5-fold. Similarly, the tryptic test set yielded an 8.8 fold increase and the chymotryptic sets averaged a 10-fold increase. The overall accuracy of GRAEZ classification (e.g., the proportion of correct assignments) averaged 0.922 for tryptic digests. Similar sensitivity and specificity was achieved for the chymotryptic species, as shown in Table 2. Furthermore, tryptic peptide and glycopeptide test sets were evaluated using the initial study which proposed a MD difference between these species. (Lehmann et al., 2000). While the original study achieved some improvement in identifying likely peptides, the TPR of glycopeptide assignment dropped to 0.68, meaning over 30% of tryptic peptides were misclassified as nonmodified peptides in silico using the original MD classification scheme. GRAEZ classification is therefore more sensitive for glycopeptides.

The GRAEZ settings were further applied in silico to the remaining set of 1197 proteins to verify their performance on another data set. Both the tryptic and chymotryptic test sets gave a negligible change in accuracy in the training set (Table 2), which may indicate that the GRAEZ classifier is robust. In total, over 100,000 tryptic species were tested in silico and GRAEZ correctly classified 91.9% of these species. Similar accuracy was achieved with the chymotryptic test set species (93.3%), which numbered >90,000.

The in silico training sets were also evaluated as the ¹³C₁ and ¹³C₂ isotope, in addition to the monoisotopic species. The GRAEZ classification did not change with the heavy isotopes over 99% of the time, which may be useful for larger analytes for which the ¹³C₁ or ¹³C₂ isotopes are abundant. The experimental data shown in Table 3 also support this assumption, as the majority of glycopeptide precursors in human urine had at least one isotopic shift (Table 3). Combinatorial approaches to glycoproteomics assign glycopeptides by matching experimentally observed monoisotopic m/z values to a combination of a glycan and a peptide mass.

TABLE 2
A summary of the testing outcomes for the in silico data. Entries are separated by
Species, Training/Test dataset (Dataset); Protease; GRAEZ classification (Glycopeptide
or Peptide); false/true positive rate (FPR/TPR), number of species (n); and the accuracy
of the test. Correct assignments are underlined, and the overall accuracy of the GRAEZ
classifier on each dataset is bolded.

| | | | GRAEZ | GRAEZ | | | |
|---|---|---|---|---|---|---|---|
| Species | Dataset | Protease | Glycopeptide | Peptide | FPR/TPR | n | Accuracy |
| Peptide | Training | Trypsin | 2,502 | 24,952 | 0.100 | 24,952 | 0.926 |
| Glycopeptide | Training | Trypsin | 23,741 | 1,205 | 0.952 | 24,946 | |
| Peptide | Test | Trypsin | 5,978 | 49,144 | 0.108 | 55,122 | 0.919 |
| Glycopeptide | Test | Trypsin | 50,327 | 2,835 | 0.947 | 53,162 | |
| Peptide | Training | Chymotrypsin | 1,676 | 18,618 | 0.083 | 20,294 | 0.934 |
| Glycopeptide | Training | Chymotrypsin | 16,839 | 817 | 0.954 | 17,656 | |
| Peptide | Test | Chymotrypsin | 4,335 | 44,338 | 0.089 | 48,673 | 0.933 |
| Glycopeptide | Test | Chymotrypsin | 40,184 | 1,812 | 0.957 | 41,996 | |

TABLE 3
An annotated set of glycopeptide assignments identified by LC-MS/MS. A total of
64 species were assigned, and relevant analytical information has been tabulated. A high
degree of sialylated glycopeptides were observed with 1-3 sialic acid residues, and a total of 23
distinct glycan compositions were observed. For the Glycan Composition entry, the following
notations were used: H, Hexose; N,N-acetylHexosamine; F, fucose; A,N-acetyl neuraminic
acid. Each glycan assignment was supported by a sub −20 ppm mass error in the MS/MS
spectra.

| | | | | | | | | | ppm |
|---|---|---|---|---|---|---|---|---|---|
| | | | | | | | | | error |
| | | | | | | | Glycan | | for |
| OFFGEL | RT | Precursor | | | Glycan | Peptide | fragment | | glycan |
| Fraction | (MIN) | m/z | z | MH+ | Composition | MH+ | mass | # 13C | loss |
| 1 | 5.0 | 1041.3996 | 4 | 4162.5766 | H6N5A3 | 1301.6098 | 2862.9702 | 2 | −12.7 |
| 1 | 9.8 | 949.3801 | 4 | 3794.4986 | H5N4A2 | 1588.7300 | 2205.7700 | 1 | −2.4 |
| 1 | 19.4 | 1025.4285 | 3 | 3074.2710 | H5N4A2 | 868.5042 | 2205.7668 | 1 | −3.9 |
| 1 | 21.9 | 961.9293 | 4 | 3844.6954 | H5N4A2 | 1638.9270 | 2205.7730 | 1 | −1.1 |
| 2 | 5.4 | 960.6302 | 4 | 3839.4990 | H5N4A2 | 1632.7377 | 2206.7613 | 2 | −7.9 |
| 2 | 35.6 | 1375.9322 | 3 | 4125.7821 | H5N4F1A1 | 2065.0513 | 2060.7308 | 1 | −0.3 |
| 5 | 9.2 | 1001.4104 | 3 | 3002.2167 | H5N4A2 | 796.4458 | 2205.7709 | 1 | −2.0 |
| 5 | 9.3 | 1048.7663 | 3 | 3144.2844 | H5N4A2 | 937.5064 | 2206.7780 | 2 | −0.3 |
| 5 | 15.7 | 1088.4593 | 3 | 3263.3634 | H7N4A1 | 1025.6070 | 2237.7564 | 0 | −11.9 |
| 6 | 4.9 | 877.5990 | 4 | 3507.3742 | H5N4A2 | 1301.6400 | 2205.7342 | 1 | −18.7 |
| 6 | 7.4 | 935.6323 | 4 | 3739.5074 | H5N4F1A2 | 1386.6700 | 2352.8374 | 2 | 3.3 |
| 6 | 11.0 | 960.3997 | 4 | 3838.5770 | H5N4F1A2 | 1485.7400 | 2352.8370 | 2 | 3.1 |
| 6 | 13.5 | 980.7674 | 3 | 2940.2877 | H5N4A1 | 1025.6100 | 1914.6777 | 1 | 3.8 |
| 6 | 14.1 | 1055.7649 | 3 | 3165.2802 | H5N4A2 | 959.5118 | 2205.7684 | 1 | −3.2 |
| 6 | 16.1 | 1078.1338 | 3 | 3232.3869 | H5N4F2A1 | 1025.6093 | 2206.7776 | 1 | −2.2 |
| 6 | 16.2 | 1126.4807 | 3 | 3377.4276 | H5N4F1A2 | 1025.6100 | 2351.8176 | 1 | −3.9 |
| 6 | 16.9 | 1029.9271 | 4 | 4116.6866 | H5N4F1A2 | 1763.8567 | 2352.8299 | 2 | 0.1 |
| 7 | 9.9 | 1126.4680 | 3 | 3377.3895 | H5N4F1A1 | 1316.6485 | 2060.7410 | 1 | 4.7 |
| 7 | 12.0 | 997.1022 | 3 | 2989.2921 | H6N3A1 | 1115.6331 | 1873.6590 | 1 | 3.0 |
| 7 | 12.1 | 943.0851 | 3 | 2827.2408 | H5N3A1 | 1115.6363 | 1711.6045 | 1 | 1.8 |
| 7 | 13.5 | 938.9036 | 4 | 3752.5926 | H5N4A2 | 1546.8113 | 2205.7813 | 1 | 2.7 |
| 7 | 14.1 | 1055.4285 | 3 | 3164.2710 | H5N4A2 | 959.5068 | 2204.7642 | 0 | −3.6 |
| 7 | 19.5 | 995.9446 | 4 | 3980.7566 | H5N4A2 | 1775.9700 | 2204.7866 | 0 | 6.6 |
| 7 | 20.4 | 1027.4584 | 4 | 4106.8118 | H5N4A2 | 1902.0600 | 2204.7518 | 0 | −9.2 |
| 7 | 21.8 | 1003.9319 | 4 | 4012.7058 | H5N4A2 | 1806.9400 | 2205.7658 | 1 | −4.3 |
| 7 | 23.2 | 988.7003 | 4 | 3951.7794 | H5N4A1 | 2037.1035 | 1914.6759 | 1 | 2.9 |
| 7 | 24.6 | 1061.2232 | 4 | 4241.8710 | H5N4A2 | 2037.0967 | 2204.7743 | 0 | 1.0 |
| 7 | 26.1 | 912.0044 | 5 | 4555.9929 | H5N4A2 | 2349.2200 | 2206.7729 | 2 | −2.6 |
| 7 | 28.5 | 1034.2194 | 4 | 4133.8558 | H5N4A2 | 1927.0600 | 2206.7958 | 2 | 7.7 |
| 9 | 9.4 | 767.9207 | 5 | 3835.5744 | H5N5A2 | 1425.7300 | 2409.8444 | 2 | −5.9 |
| 10 | 6.1 | 935.1452 | 4 | 3737.5590 | H5N4A2 | 1530.7865 | 2206.7725 | 2 | −2.8 |
| 10 | 6.6 | 787.1215 | 5 | 3931.5784 | H5N4A2 | 1725.8100 | 2205.7684 | 1 | −3.1 |
| 10 | 26.6 | 914.4295 | 4 | 3654.6962 | H5N4A2 | 1448.9200 | 2205.7762 | 1 | 0.4 |
| 16 | 7.2 | 1073.4713 | 3 | 3218.3994 | H5N4F1A1 | 1158.6700 | 2059.7294 | 0 | 0.7 |
| 16 | 8.6 | 1007.7892 | 3 | 3021.3531 | H5N4F1A1 | 961.6200 | 2059.7331 | 0 | 2.5 |
| 16 | 13.9 | 1029.4527 | 3 | 3086.3436 | H5N4F1A1 | 1024.6000 | 2061.7436 | 2 | 4.3 |
| 16 | 15.5 | 1047.9351 | 4 | 4188.7186 | H6N5F1A1 | 1762.8487 | 2425.8699 | 1 | 2.7 |
| 16 | 15.6 | 956.4007 | 4 | 3822.5810 | H5N4F1A1 | 1762.8530 | 2059.7280 | 0 | 0.0 |
| 16 | 27.4 | 1085.1565 | 3 | 3253.4550 | H5N4F1A1 | 1193.7246 | 2059.7304 | 0 | 1.1 |
| 16 | 28.2 | 909.6679 | 4 | 3635.6498 | H5N4F1A1 | 1574.9200 | 2060.7298 | 1 | −0.8 |
| 16 | 30.6 | 1291.9146 | 3 | 3873.7293 | H5N4F1A1 | 1812.9990 | 2060.7303 | 1 | −0.5 |
| 16 | 32.1 | 962.6926 | 4 | 3847.7486 | H6N5F1A1 | 1422.8871 | 2424.8615 | 0 | 0.6 |
| 16 | 32.5 | 977.4430 | 4 | 3906.7502 | H6N6A1 | 1422.8785 | 2483.8717 | 2 | −9.7 |
| 16 | 32.6 | 936.6859 | 4 | 3743.7218 | H5N6A1 | 1422.8845 | 2320.8373 | 1 | −0.9 |
| 16 | 32.9 | 1229.9039 | 3 | 3687.6972 | H5N5F1A1 | 1422.8881 | 2264.8091 | 2 | −2.0 |
| 16 | 32.9 | 929.9304 | 4 | 3716.6998 | H5N3F2A2 | 1422.8800 | 2293.8198 | 0 | 10.8 |
| 16 | 32.9 | 934.6821 | 4 | 3735.7066 | H5N4F1A1 | 1673.9700 | 2061.7366 | 2 | 0.9 |
| 16 | 34.4 | 873.6599 | 4 | 3491.6178 | H5N4F1 | 1721.9700 | 1769.6478 | 1 | 7.0 |
| 16 | 36.4 | 946.6840 | 4 | 3783.7142 | H5N4F1A1 | 1721.9600 | 2061.7542 | 2 | 9.5 |
| 17 | 7.5 | 821.1278 | 4 | 3281.4894 | H5N4F2A1 | 1074.7200 | 2206.7694 | 1 | −9.0 |
| 17 | 13.7 | 865.3820 | 4 | 3458.5062 | H5N5F2 | 1339.7295 | 2118.7767 | 1 | 4.9 |
| 17 | 13.8 | 1104.4873 | 3 | 3311.4474 | H5N5F1 | 1339.7293 | 1971.7181 | 0 | 3.1 |
| 17 | 14.0 | 920.4036 | 4 | 3678.5926 | H6N6F1 | 1339.7300 | 2338.8626 | 2 | 5.1 |
| 17 | 14.2 | 883.8876 | 4 | 3532.5286 | H6N6 | 1339.7259 | 2192.8027 | 2 | 15.0 |
| 17 | 15.4 | 993.1662 | 4 | 3969.6430 | H5N4F2A1 | 1762.8503 | 2206.7927 | 1 | 1.5 |
| 17 | 17.0 | 1009.7729 | 3 | 3027.3042 | H5N5F1 | 1054.5865 | 1972.7177 | 1 | 1.2 |
| 17 | 37.6 | 1020.7279 | 4 | 4079.8898 | H6N6F1 | 1742.0423 | 2337.8475 | 1 | 0.1 |
| 18 | 9.6 | 898.9125 | 4 | 3592.6282 | H5N4F2A1 | 1386.8573 | 2205.7709 | 0 | −3.7 |
| 19 | 11.7 | 878.4148 | 4 | 3510.6374 | H5N4A2 | 1303.8825 | 2206.7549 | 2 | −10.8 |
| 23 | 14.3 | 1058.6978 | 4 | 4231.7694 | H5N6F3 | 1762.8571 | 2468.9123 | 2 | 5.1 |
| 23 | 14.6 | 971.1642 | 4 | 3881.6350 | H5N5F2 | 1762.8519 | 2118.7831 | 1 | 7.9 |

### GRAEZ Evaluation of Published Reports

To further validate the in silico results, published proteomic and glycoproteomic data were also evaluated. GRAEZ testing of a recently published proteomic data set of the HeLa cell proteome (Nagaraj et al., 2011) correctly classified 96.2% of 4,760 unique tryptic peptides between 1500 and 5000 Daltons as peptides, with a specificity of 0.962. Similarly, a retrospective GRAEZ classification of several published site-specific glycoproteomic studies was also performed to validate the sensitivity of the method. As glycoproteomics studies have not approached the scale of proteomics studies, results of several studies were utilized to generate a data set comprising glycopeptides for testing the GRAEZ classification. These studies examined a variety of different samples, including glycoprotein standards (Hart-Smith and Raftery, 2012), fetal bovine serum (Wang et al., 2010), human urine (Halim et al., 2011), murine zona pellucida glycoproteins (Goldberg et al., 2007), human haptoglobin (Wang et al., 2011), human alpha-1 acid glycoprotein (Zhang et al., 2008), hepatitis C glycoprotein (Iacob et al., 2008), HIV envelope glycoprotein gp140 (Irungu et al., 2008), and human IgG subclasses (Wuhrer et al., 2007). Thus, 624 nonredundant, intact tryptic glycopeptides were identified in these studies within the mass range of 1500-5000 Daltons. Subsequent GRAEZ testing was performed on experimental m/z values when given, and on imputed m/z values when absent. GRAEZ correctly classified 564 of these species as glycopeptides for an overall sensitivity of 0.904. This result demonstrates that the sensitivity of GRAEZ classification was maintained among these reports on diverse samples. Accordingly, analysis of data from multiple organisms obtained using different platforms demonstrated that GRAEZ classification may be useful for a variety of now known and future N-glycoproteomic studies to identify likely N-glycopeptide precursors in LC-MS.

### Experimental Validation of GRAEZ Classification

The utility of GRAEZ was further evaluated experimentally using tryptic peptides isolated from urine. Urine is a highly complex, clinically relevant sample type, and contains numerous salts, peptides and metabolites. To combat the possibility of non-peptide background contamination affecting the classification, peptides were labeled with amine-reactive TMT tags before analysis. Using the mzpresent tool, every MS² spectra collected was searched for two fragment ions: the TMT reporter tag at 126.1277 which was required for “peptide” designation, and the 366.1395 peak, which was required for “glycopeptide” designation. Species without either of these ions were not considered in the GRAEZ classification.

Urine was chosen by way of experiment because it is a highly complex sample containing thousands of proteins. In addition, by way of experiment, glycopeptide enrichment was not performed, to access performance of the GRAEZ classifier.

An analysis of MS² spectra (n=90,624) showed that 90% (692/772) of all species that yielded oxonium fragments upon activation by HCD were characterized as N-glycopeptides by the GRAEZ algorithm. Similarly, 93% (83,289/89,852) of all peptide species were correctly classified. In total, 116 unique peptide precursors were selected by DDA for every glycopeptide precursor. In addition, the samples were analyzed using peptide-optimized MS settings, and there was a majority (>85%) of low-quality spectra acquired. Few studies intentionally analyze intact glycopeptides and peptides simultaneously, since peptides and glycopeptides have distinct optimal instrumental parameters. (Krenyacz et al., 2009; Froehlich et al., 2011).

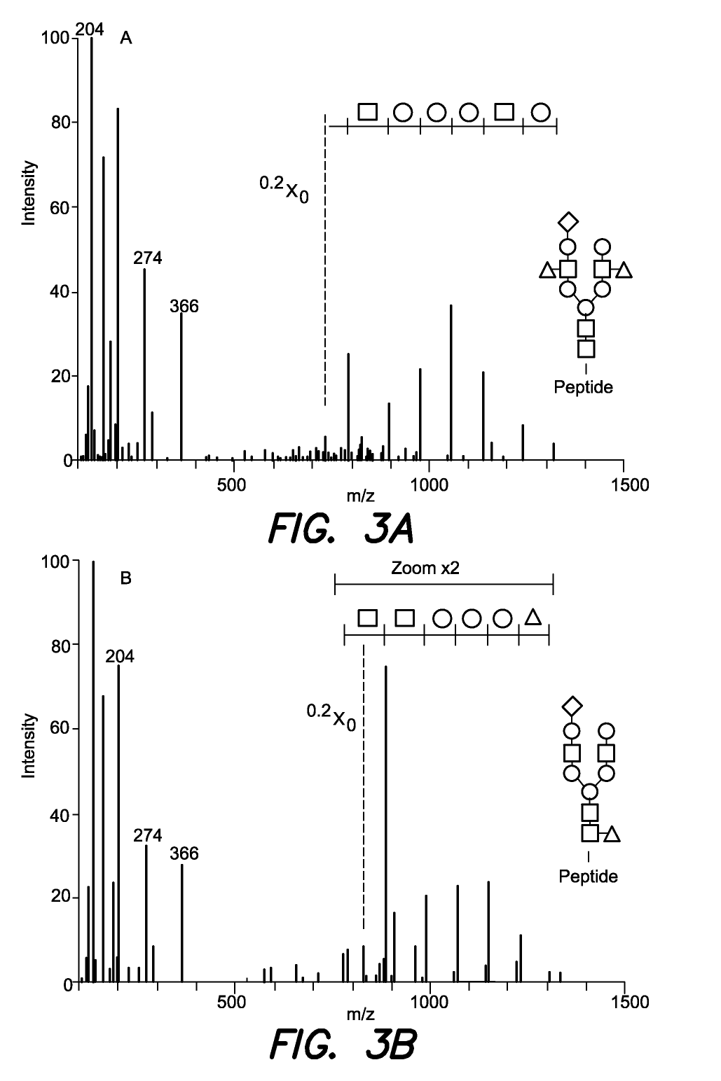

Several high-quality glycopeptide fragmentations were observed, and the glycan portions were assigned by the presence of the abundant Y₁ ion (nomenclature as described in Domon and Costello, 1988) and a minimum of three other glycosidic fragment ions. Two examples of higher quality spectra are shown in FIGS. 3A and 3B. In each spectrum, a loss corresponding to the nonreducing end glycan moieties was observed, followed by successive losses of 6 monosaccharide residues. In both spectra, a ^{0.2}X₀ ion was observed and in FIG. 3B, loss of the terminal GlcNac residue was also observed. Each spectrum identified the mass of the peptide portion in addition to the glycan composition. Spectra corresponding to a total of 61 glycopeptides were acquired with sufficient quality to manually assign the glycan portion of the glycopeptides in the data-dependent analyses, and relevant information is shown in Table 3. These species were predominantly glycopeptides with sialylated complex-type glycans. The peptide MH⁺ values were imputed after assignment of the MS/MS pattern observed, usually supported by abundant Y₁ and ^{0.2}X₀ type ions. After identifying the Y₁ ion, the remaining mass lost from the calculated precursor MH+ was determined and cross referenced against plausible N-glycan compositions to confirm the compositional assignment. Each glycan loss matched an N-glycan composition at less than 20 ppm mass tolerance. By way of example, the peptide portions were not sequenced in the present study, and are reported as their imputed (M+H)⁺ values.

### Prospective Analysis of Precursors of Interest

An unfractionated sample of urinary peptides was initially analyzed by DDA MS/MS and subsequently by targeted MS. A total of 2,325 species from the initial analysis were characterized as glycopeptides by the GRAEZ. A total of 3,196 MS² spectra were acquired, and 2,598 (81%) of these had an oxonium ion at a minimum of 25% of the base peak intensity. A less stringent cutoff of 5% increases the number to 2878, or 90% of all MS² spectra acquired. Our fractionated urine sample gave a glycopeptide sampling rate of only 0.8% by comparison, generating only 772 MS² spectra in substantially more instrument time. Therefore, generating a targeted list based on GRAEZ classification significantly increased both the glycopeptide MS/MS sampling efficiency and sensitivity.

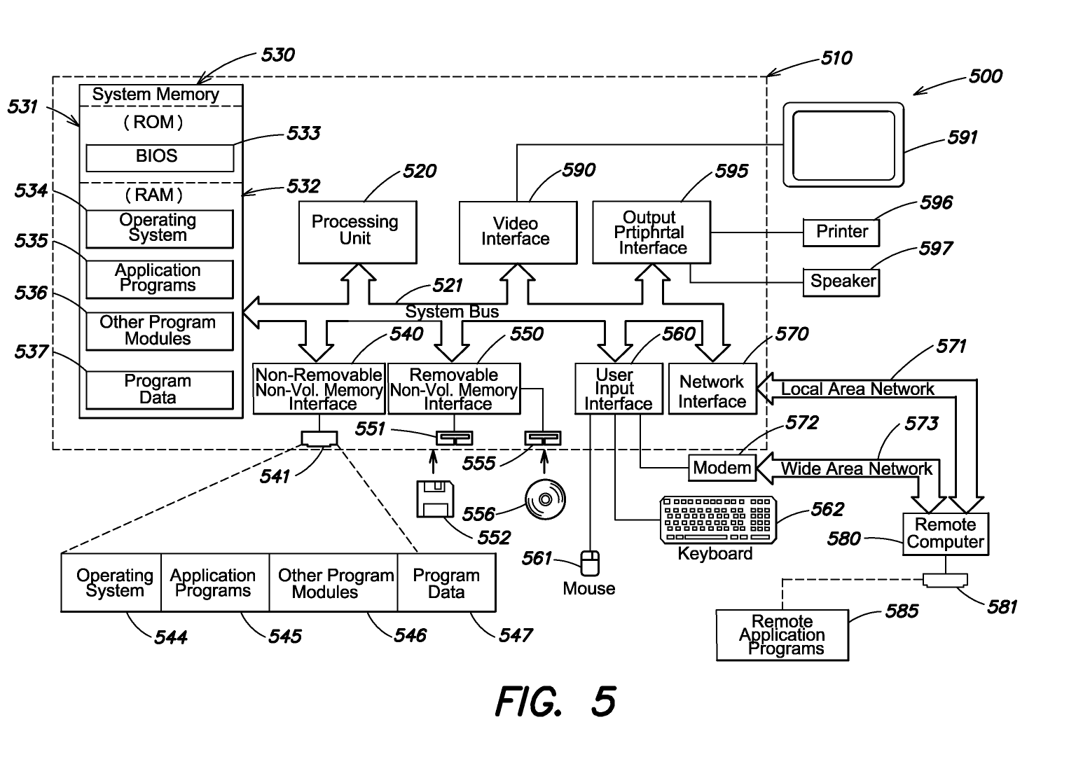

FIG. 5 illustrates an example of a suitable computing system environment 500 on which the disclosed technology may be implemented. The computing system environment 500 is only one example of a suitable computing environment and is not intended to suggest any limitation as to the scope of use or functionality of the disclosed technology. Neither should the computing environment 500 be interpreted as having any dependency or requirement relating to any one or combination of components illustrated in the exemplary operating environment 500.

Embodiments are operational with numerous other general purpose or special purpose computing system environments or configurations. Examples of well-known computing systems, environments, and/or configurations that may be suitable for use with the disclosed technology include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The computing environment may execute computer-executable instructions, such as program modules. Generally, program modules include routines, programs, objects, components, data structures, etc. that perform particular tasks or implement particular abstract data types. Embodiments may also be practiced in distributed computing environments where tasks are performed by remote processing devices that are linked through a communications network. In a distributed computing environment, program modules may be located in both local and remote computer storage media including memory storage devices.

With reference to FIG. 5, an exemplary system for implementing the embodiment includes a general purpose computing device in the form of a computer 510. Components of computer 510 may include, but are not limited to, a processing unit 520, a system memory 530, and a system bus 521 that couples various system components including the system memory to the processing unit 520. The system bus 521 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 510 typically includes a variety of computer readable media. Computer readable media can be any available media that can be accessed by computer 510 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 510. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 530 includes computer storage media in the form of volatile and/or nonvolatile memory such as read only memory (ROM) 531 and random access memory (RAM) 532. A basic input/output system 533 (BIOS), containing the basic routines that help to transfer information between elements within computer 510, such as during start-up, is typically stored in ROM 531. RAM 532 typically contains data and/or program modules that are immediately accessible to and/or presently being operated on by processing unit 520. By way of example, and not limitation, FIG. 5 illustrates operating system 534, application programs 535, other program modules 536, and program data 537.

The computer 510 may also include other removable/non-removable, volatile/nonvolatile computer storage media. By way of example only, FIG. 5 illustrates a hard disk drive 541 that reads from or writes to non-removable, nonvolatile magnetic media, a magnetic disk drive 551 that reads from or writes to a removable, nonvolatile magnetic disk 552, and an optical disk drive 555 that reads from or writes to a removable, nonvolatile optical disk 556 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 541 is typically connected to the system bus 521 through an non-removable memory interface such as interface 540, and magnetic disk drive 551 and optical disk drive 555 are typically connected to the system bus 521 by a removable memory interface, such as interface 550.

The drives and their associated computer storage media discussed above and illustrated in FIG. 5, provide storage of computer readable instructions, data structures, program modules and other data for the computer 510. In FIG. 5, for example, hard disk drive 541 is illustrated as storing operating system 544, application programs 545, other program modules 546, and program data 547. Note that these components can either be the same as or different from operating system 534, application programs 535, other program modules 536, and program data 537. Operating system 544, application programs 545, other program modules 546, and program data 547 are given different numbers here to illustrate that, at a minimum, they are different copies. A user may enter commands and information into the computer 510 through input devices such as a keyboard 562 and pointing device 561, commonly referred to as a mouse, trackball or touch pad. Other input devices (not shown) may include a microphone, joystick, game pad, satellite dish, scanner, or the like. These and other input devices are often connected to the processing unit 520 through a user input interface 560 that is coupled to the system bus, but may be connected by other interface and bus structures, such as a parallel port, game port or a universal serial bus (USB). A monitor 591 or other type of display device is also connected to the system bus 521 via an interface, such as a video interface 590. In addition to the monitor, computers may also include other peripheral output devices such as speakers 597 and printer 596, which may be connected through a output peripheral interface 595.

The computer 510 may operate in a networked environment using logical connections to one or more remote computers, such as a remote computer 580. The remote computer 580 may be a personal computer, a server, a router, a network PC, a peer device or other common network node, and typically includes many or all of the elements described above relative to the computer 510, although only a memory storage device 581 has been illustrated in FIG. 5. The logical connections depicted in FIG. 5 include a local area network (LAN) 571 and a wide area network (WAN) 573, but may also include other networks. Such networking environments are commonplace in offices, enterprise-wide computer networks, intranets and the Internet.

When used in a LAN networking environment, the computer 510 is connected to the LAN 571 through a network interface or adapter 570. When used in a WAN networking environment, the computer 510 typically includes a modem 572 or other means for establishing communications over the WAN 573, such as the Internet. The modem 572, which may be internal or external, may be connected to the system bus 521 via the user input interface 560, or other appropriate mechanism. In a networked environment, program modules depicted relative to the computer 510, or portions thereof, may be stored in the remote memory storage device. By way of example, and not limitation, FIG. 5 illustrates remote application programs 585 as residing on memory device 581. It will be appreciated that the network connections shown are exemplary and other means of establishing a communications link between the computers may be used.

The above-described embodiments may be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the disclosed technology may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the disclosed technology as discussed above. As used herein, the term “computer-readable storage medium” encompasses only a computer-readable medium that can be considered to be a manufacture (i.e., article of manufacture) or a machine. Alternatively or additionally, the disclosed technology may be embodied as a computer readable medium other than a computer-readable storage medium, such as a propagating signal.

The terms “program” or “software” are used herein in a generic sense to refer to any type of computer code or set of computer-executable instructions that can be employed to program a computer or other processor to implement various aspects of the embodiments as discussed above. Additionally, it should be appreciated that according to one aspect of this embodiment, one or more computer programs that when executed perform methods of the present disclosed technology need not reside on a single computer or processor, but may be distributed in a modular fashion amongst a number of different computers or processors to implement various aspects of the present disclosed technology.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the embodiments may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, some aspects of the disclosed technology may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having thus described several aspects of at least one embodiment of the disclosed technology, it is to be appreciated that various alterations, modifications, and improvements will readily occur to those skilled in the art.

The described techniques may be implemented in software, hardware, firmware, circuitry, or any combination thereof. As discussed above, in some embodiments, the tool may be implemented as computer-readable instructions stored on one or more non-transitory computer-readable media. The computer-readable instructions, when executed by one or more processors, may cause a computing device to perform the described method of discriminating between peptides and glycopeptides in a sample. Results of the discrimination may be further processed, analyzed, stored, presented to a user in a suitable manner on a suitable user interface, or otherwise manipulated. In some embodiments, the glycopeptides identified in the sample may be further analyzed and it may be determined which glycan moieties occupy specific glycosylation sites.

Furthermore, the described techniques may be incorporated into any suitable system. For example, the tool may be executed by a system performing mass spectrometry (e.g., tandem mass spectrometry), which may be a system performing an entire analysis of a sample or a system or a device performing any one or more steps of the mass spectrometry analysis. Further, the described techniques may be incorporated into a system or device performing data-dependent acquisition (DDA).

Such alterations, modifications, and improvements are intended to be part of this disclosure, and are intended to be within the spirit and scope of some embodiments. Further, though advantages of some embodiments are indicated, it should be appreciated that not every embodiment of the disclosed technology will include every described advantage. Some embodiments may not implement any features described as advantageous herein and in some instances. Accordingly, the foregoing description and drawings are by way of example only.

### REFERENCES

1. Varki, A. (1993) Biological roles of oligosaccharides: all of the theories are correct. Glycobiology 3, 97-130.
2. An, H. J., Froehlich, J. W., and Lebrilla, C. B. (2009) Determination of glycosylation sites and site-specific heterogeneity in glycoproteins. Curr Opin Chem Biol 13, 421-426.
3. Dodds, E. D. (2012) Gas-phase dissociation of glycosylated peptide ions. Mass Spectrom Rev 31, 666-682.
4. Dalpathado, D. S., Irungu, J., Go, E. P., Butnev, V. Y., Norton, K., Bousfield, G. R., and Desaire, H. (2006) Comparative glycomics of the glycoprotein follicle stimulating hormone: glycopeptide analysis of isolates from two mammalian species. Biochemistry 45, 8665-8673.
5. Clowers, B. H., Dodds, E. D., Seipert, R. R., and Lebrilla, C. B. (2007) Site determination of protein glycosylation based on digestion with immobilized nonspecific proteases and Fourier transform ion cyclotron resonance mass spectrometry. J Proteome Res 6, 4032-4040.
6. Kolarich, D., Jensen, P. H., Altmann, F., and Packer, N. H. (2012) Determination of site-specific glycan heterogeneity on glycoproteins. Nat Protoc 7, 1285-1298.
7. Desaire, H., and Hua, D. (2009) When can glycopeptides be assigned based solely on high-resolution mass spectrometry data? International Journal of Mass Spectrometry 287, 21-26.
8. Ito, S., Hayama, K., and Hirabayashi, J. (2009) Enrichment strategies for glycopeptides. Methods Mol Biol 534, 195-203.
9. Huddleston, M. J., Bean, M. F., and Carr, S. A. (1993) Collisional fragmentation of glycopeptides by electrospray ionization LC/MS and LC/MS/MS: methods for selective detection of glycopeptides in protein digests. Anal Chem 65, 877-884.
10. Jebanathirajah, J., Steen, H., and Roepstorff, P. (2003) Using optimized collision energies and high resolution, high accuracy fragment ion selection to improve glycopeptide detection by precursor ion scanning. J Am Soc Mass Spectrom 14, 777-784.
11. Bruce, C., Shifman, M. A., Miller, P., and Gulcicek, E. E. (2006) Probabilistic enrichment of phosphopeptides by their mass defect. Anal Chem 78, 4374-4382.
12. Dodds, E. D., An, H. J., Hagerman, P. J., and Lebrilla, C. B. (2006) Enhanced peptide mass fingerprinting through high mass accuracy: Exclusion of non-peptide signals based on residual mass. J Proteome Res 5, 1195-1203.
13. Kirchner, M., Timm, W., Fong, P., Wangemann, P., and Steen, H. (2010) Non-linear classification for on-the-fly fractional mass filtering and targeted precursor fragmentation in mass spectrometry experiments. Bioinformatics 26, 791-797.
14. Lehmann, W. D., Bohne, A., and von Der Lieth, C. W. (2000) The information encrypted in accurate peptide masses-improved protein identification and assistance in glycopeptide identification and characterization. J Mass Spectrom 35, 1335-1341.
15. Vaezzadeh, A. R., Briscoe, A. C., Steen, H., and Lee, R. S. (2010) One-step sample concentration, purification, and albumin depletion method for urinary proteomics. J Proteome Res 9, 6082-6089.
16. Cox, J., and Mann, M. (2008) MaxQuant enables high peptide identification rates, individualized p.p.b.-range mass accuracies and proteome-wide protein quantification. Nat Biotechnol 26, 1367-1372.
17. Nagaraj, N., Wisniewski, J. R., Geiger, T., Cox, J., Kircher, M., Kelso, J., Paabo, S., and Mann, M. (2011) Deep proteome and transcriptome mapping of a human cancer cell line. Mol Syst Biol 7, 548.
18. Hart-Smith, G., and Raftery, M. J. (2012) Detection and characterization of low abundance glycopeptides via higher-energy C-trap dissociation and orbitrap mass analysis. J Am Soc Mass Spectrom 23, 124-140.
19. Wang, X., Emmett, M. R., and Marshall, A. G. (2010) Liquid chromatography electrospray ionization Fourier transform ion cyclotron resonance mass spectrometric characterization of N-linked glycans and glycopeptides. Anal Chem 82, 6542-6548.
20. Halim, A., Nilsson, J., Ruetschi, U., Hesse, C., and Larson, G. (2011) Human urinary glycoproteomics; attachment site specific analysis of N- and O-linked glycosylations by CID and ECD. Mol Cell Proteomics.
21. Goldberg, D., Bern, M., Parry, S., Sutton-Smith, M., Panico, M., Morris, H. R., and Dell, A. (2007) Automated N-glycopeptide identification using a combination of single- and tandem-MS. J Proteome Res 6, 3995-4005.
22. Wang, D., Hincapie, M., Rejtar, T., and Karger, B. L. (2011) Ultrasensitive characterization of site-specific glycosylation of affinity-purified haptoglobin from lung cancer patient plasma using 10 mum i.d. porous layer open tubular liquid chromatography-linear ion trap collision-induced dissociation/electron transfer dissociation mass spectrometry. Anal Chem 83, 2029-2037.
23. Zhang, Y., Go, E. P., and Desaire, H. (2008) Maximizing coverage of glycosylation heterogeneity in MALDI-MS analysis of glycoproteins with up to 27 glycosylation sites. Anal Chem 80, 3144-3158.
24. Iacob, R. E., Perdivara, I., Przybylski, M., and Tomer, K. B. (2008) Mass spectrometric characterization of glycosylation of hepatitis C virus E2 envelope glycoprotein reveals extended microheterogeneity of N-glycans. J Am Soc Mass Spectrom 19, 428-444.
25. Irungu, J., Go, E. P., Zhang, Y., Dalpathado, D. S., Liao, H. X., Haynes, B. F., and Desaire, H. (2008) Comparison of HPLC/ESI-FTICR MS versus MALDI-TOF/TOF MS for glycopeptide analysis of a highly glycosylated HIV envelope glycoprotein. J Am Soc Mass Spectrom 19, 1209-1220.
26. Wuhrer, M., Stam, J. C., van de Geijn, F. E., Koeleman, C. A., Verrips, C. T., Dolhain, R. J., Hokke, C. H., and Deelder, A. M. (2007) Glycosylation profiling of immunoglobulin G (IgG) subclasses from human serum. Proteomics 7, 4070-4081.
27. Krenyacz, J., Drahos, L., and Vekey, K. (2009) Letter: Collision energy and cone voltage optimisation for glycopeptide analysis. Eur J Mass Spectrom (Chichester, Eng) 15, 361-365.
28. Froehlich, J. W., Barboza, M., Chu, C., Lerno, L. A., Clowers, B. H., Zivkovic, A. M., German, J. B., and Lebrilla, C. B. (2011) Nano-LC-MS/MS of Glycopeptides Produced by Nonspecific Proteolysis Enables Rapid and Extensive Site-Specific Glycosylation Determination. Anal Chem 83, 5541-5547.
29. Domon, B., and Costello, C. E. (1988) A systematic nomenclature for carbohydrate fragmentations in FAB-MS/MS spectra of glycoconjugates. Glycoconjugate Journal 5, 397-409.
30. Toumi, M. L., and Desaire, H. (2010) Improving mass defect filters for human proteins. J Proteome Res 9, 5492-5495.

What is claimed is:

1. At least one computer-readable storage medium storing computer-executable instructions that, when executed by at least one processor, perform a method of identifying glycopeptides in a sample, the method comprising:
analyzing a mass spectrum of the sample to identify at least one portion of the mass spectrum having at least one characteristic of mass spectra indicative of presence of glycopeptides; and
identifying the glycopeptides in the sample based on the at least one identified portion.

2. The at least one computer-readable storage medium of claim 1, wherein the method further comprises:
determining the at least one characteristic of mass spectra indicative of presence of glycopeptides.

3. The at least one computer-readable storage medium of claim 2, wherein:
determining the at least one characteristic comprises determining at least one glycopeptide-rich acquisition enhancement zone.

4. The at least one computer-readable storage medium of claim 2, wherein:
determining the at least one characteristic comprises analyzing a training data set comprising a plurality of mass spectra of peptides.

5. The at least one computer-readable storage medium of claim 1, wherein:
the at least one characteristic comprises at least one first range of a nominal mass and at least one second range of mass defect.

6. The at least one computer-readable storage medium of claim 1, wherein the method further comprises:
displaying on a user interface results of the identification of the glycopeptides in the sample.

7. The at least one computer-readable storage medium of claim 6, wherein:
displaying the results of the identification of the glycopeptides comprises displaying the results so that the glycopeptides in the sample are differentiated from peptides in the sample.

8. The at least one computer-readable storage medium of claim 1, wherein the method further comprises:
providing a representation of the results of the identification of the glycopeptides so that the representation is enabled to receive input indicating selection of at least one glycopeptide of the identified glycopeptides for further analysis.

9. The at least one computer-readable storage medium of claim 8, wherein the method further comprises:
further analyzing the at least one glycopeptide selected for the further analysis.

10. The at least one computer-readable storage medium of claim 1, wherein:
identifying the glycopeptides in the sample comprises identifying N-glycosylated glycopeptides.

11. The at least one computer-readable storage medium of claim 1, wherein the method further comprises:
providing results of the identification of the glycopeptides in the sample to a system configured to further analyze the identified glycopeptides.

12. The at least one computer-readable storage medium of claim 1, wherein the method further comprises:
further analyzing at least one of the identified glycopeptides.

13. The at least one computer-readable storage medium of claim 1, wherein the sample comprises a biological sample.

14. The at least one computer-readable storage medium of claim 13, wherein the biological sample is obtained from tissue, urine, blood, plasma, serum or saliva.

15. The at least one computer-readable storage medium of claim 2, wherein:
the at least one characteristic is determined for a protease used to generate a mixture of peptides and glycopeptides from the sample.

16. The at least one computer-readable storage medium of claim 1, wherein:
analyzing the mass spectrum comprises analyzing precursor ion data.

17. At least one computer-readable storage medium storing computer-executable instructions that, when executed by at least one processor, perform a method of identifying glycopeptides in a sample, the method comprising:
determining at least one characteristic of mass spectra indicative of presence of glycopeptides;
analyzing a mass spectrum of the sample to identify at least one portion of the mass spectrum having the at least one characteristic; and
identifying the glycopeptides in the sample based on the at least one identified portion.

18. A computer-implemented method of identifying glycopeptides in a sample, the method comprising:
with at least one processor:
analyzing a mass spectrum of the sample to identify at least one portion of the mass spectrum having at least one characteristic of mass spectra indicative of presence of glycopeptides; and
identifying the glycopeptides in the sample based on the at least one identified portion.

19. The method of claim 18, further comprising:
determining the at least one characteristic of mass spectra indicative of presence of glycopeptides.

20. The method of claim 19, wherein:
determining the at least one characteristic comprises determining at least one glycopeptide-rich acquisition enhancement zone.

21. The method of claim 19, wherein:
determining the at least one characteristic comprises analyzing a data set comprising a plurality of mass spectra of peptides to determine at least one first range of a nominal mass and at least one second range of mass defect indicative of presence of glycopeptides.

22. The method of claim 19, wherein:
determining the at least one characteristic comprises analyzing a training data set comprising a plurality of mass spectra of peptides.

23. The method of claim 18, wherein:
the at least one characteristic comprises at least one first range of a nominal mass and at least one second range of mass defect.

24. The method of claim 18, further comprising:
displaying on a user interface results of the identification of the glycopeptides in the sample.

25. The method of claim 24, wherein:
displaying the results of the identification of the glycopeptides comprises displaying the results so that the glycopeptides in the sample are differentiated from peptides in the sample.

26. The method of claim 18, further comprising:
providing a representation of the results of the identification of the glycopeptides so that the representation is enabled to receive input indicating selection of at least one glycopeptide of the identified glycopeptides for further analysis.

27. The method of claim 26, further comprising:
further analyzing the at least one glycopeptide selected for the further analysis.

28. The method of claim 27, wherein:
further analyzing the at least one glycopeptide selected for the further analysis comprises determining a site of glycosylation on the at least one glycopeptide.

29. The system of claim 28, wherein:
determining the site of glycosylation comprises determining a site of N-glycosylation on the at least one glycopeptide.

30. The method of claim 27, further comprising:
analyzing the at least one glycopeptide using tandem mass-spectrometry.

31. The method of claim 18, wherein:
identifying the glycopeptides in the sample comprises identifying N-glycosylated glycopeptides.

32. The method of claim 18, further comprising:
providing results of the identification of the glycopeptides in the sample to a system configured to further analyze the identified glycopeptides.

33. The method of claim 18, further comprising:
further analyzing at least one of the identified glycopeptides.

34. The method of claim 18, wherein the sample comprises a biological sample.

35. The method of claim 34, wherein the biological sample is obtained from tissue, urine, blood, plasma, serum or saliva.

36. The method of claim 18, wherein:
analyzing the mass spectrum comprises analyzing precursor ion data.

37. A device comprising at least one processor and memory storing computer-executable instructions that, when executed by the at least one processor, perform a method of identifying glycopeptides in a sample, the method comprising:
analyzing a mass spectrum of the sample to identify at least one portion of the mass spectrum having at least one characteristic of mass spectra indicative of presence of glycopeptides; and
identifying the glycopeptides in the sample based on the at least one identified portion.

38. The device of claim 37, wherein the method further comprises:
determining the at least one characteristic of mass spectra indicative of presence of glycopeptides.

39. The device of claim 38, wherein:
determining the at least one characteristic comprises determining at least one glycopeptide-rich acquisition enhancement zone.

40. The device of claim 38, wherein:
determining the at least one characteristic comprises analyzing a data set comprising a plurality of mass spectra of peptides to determine at least one first range of a nominal mass and at least one second range of mass defect indicative of presence of glycopeptides.

41. The device of claim 38, wherein:
determining the at least one characteristic comprises analyzing a training data set comprising a plurality of mass spectra of peptides.

42. A system comprising:
a device comprising at least one processor and memory storing computer-executable instructions that, when executed by the at least one processor, perform a method of identifying glycopeptides in a sample, the method comprising:
analyzing a mass spectrum of the sample to identify at least one portion of the mass spectrum having at least one characteristic of mass spectra indicative of presence of glycopeptides;
identifying the glycopeptides in the sample based on the at least one identified portion; and
analyzing at least one glycopeptide of the identified glycopeptides.

43. The system of claim 42, wherein the method further comprises:
determining the at least one characteristic of mass spectra indicative of presence of glycopeptides.

44. The system of claim 43, wherein:
determining the at least one characteristic comprises determining at least one glycopeptide-rich acquisition enhancement zone.

45. The system of claim 43, wherein:
determining the at least one characteristic comprises analyzing a data set comprising a plurality of mass spectra of peptides to determine at least one first range of a nominal mass and at least one second range of mass defect indicative of presence of glycopeptides.

46. The system of claim 43, wherein:
determining the at least one characteristic comprises analyzing a training data set comprising a plurality of mass spectra of peptides.

47. The system of claim 42, wherein:
analyzing the at least one glycopeptide comprises determining a site of glycosylation on the at least one glycopeptide.

48. The system of claim 47, wherein:
determining the site of glycosylation comprises determining a site of N-glycosylation on the at least one glycopeptide.
